(19) United States
(12) Patent Application Publication
(10) Pub. No.: US 2016/0235370 A1
(43) Pub. Date: Aug. 18, 2016

(54) SYSTEMS AND METHOD FOR DLCO AND HEMOGLOBIN MEASUREMENTS

(71) Applicants: Avi LAZAR, Ra'Anana (IL); Yoav Meiraz, Avihail (IL); Robert Shiner, Herzlia (IL)

(72) Inventors: Avi LAZAR, Ra'Anana (IL); Yoav Meiraz, Avihail (IL); Robert Shiner, Herzlia (IL)

(21) Appl. No.: 15/041,643

(22) Filed: Feb. 11, 2016

Related U.S. Application Data
(60) Provisional application No. 62/114,764, filed on Feb. 11, 2015.

(51) Int. Cl. A61B 5/00; A61B 5/091; A61B 5/145

(52) U.S. Cl. CPC A61B 5/7221; A61B 5/742; A61B 5/14546; A61B 5/091; A61B 5/7475; A61B 5/0015

(57) ABSTRACT

According to some embodiments, an Hb level value for a patient may be received from an Hb meter (e.g., a non-invasive Hb meter). Moreover, a DLCO value for the patient may be received from a DLCO analyzer at substantially the same time. A computer may then automatically calculate a predicted Hb value for the patient based on at least one patient parameter (e.g., a patient gender, age, and/or weight). If the predicted Hb value for the patient substantially matches the received Hb level value, the received DLCO value may be output. If the predicted Hb value for the patient does not substantially match the received Hb level value, a corrected DLCO value may be automatically calculated and output.

### CROSS-REFERENCE TO RELATED APPLICATIONS

This application claims the benefit of U.S. Provisional Patent Application No. 62/114,764 entitled “SYSTEMS AND METHODS FOR DLCO AND HEMOGLOBIN MEASUREMENTS” and filed Feb. 11, 2015. The entire content of that application is incorporated herein by reference.

### FIELD

The present invention relates to computer systems and more particularly to computer systems associated with DCLO and hemoglobin measurements.

### BACKGROUND

Taking a single breath uptake measurement of Carbon Monoxide (CO) in the lungs is a very common medical technique. The process of CO uptake can be simplified into two transfer or conductance properties: (1) membrane conductivity (DM), which reflects the diffusion properties of the alveolar capillary membrane; and (2) the binding of CO and Hb. The latter may be represented as the product of the CO-Hb chemical reaction rate (h) and the volume of Hb in alveolar capillary blood (Vc). Since these are conductance in series, these properties are related by:

$$\frac{1}{DLCO} = \frac{1}{DM} + \frac{1}{θ Vc}$$

Note, however, that a significant number of patients undergoing a Diffusing capacity of the Lung for CO (“DLCO”) test may suffer from Anemia. Moreover, these patients may not have updated and accurate Hb results. This may lead to DLCO results that are significantly biased, as can be seen from the formula above. That is, the readings may be much lower than normal.

It would therefore be desirable to provide systems and methods to facilitate accurate DLCO measurements in an automated, efficient, and accurate manner.

### SUMMARY

According to some embodiments, systems, methods, apparatus, computer program code and means may facilitate accurate DLCO measurements. In some embodiments, both Hb level and DLCO values may be measured simultaneously. The DLCO values may then be calculated normalized to standard Hb values. According to some embodiments, an Hb level value for a patient may be received from an Hb meter (e.g., a non-invasive Hb meter). Moreover, a DLCO value for the patient may be received from a DLCO analyzer at substantially the same time. A computer may then automatically calculate a predicted Hb value for the patient based on at least one patient parameter (e.g., a patient gender, age, and/or weight). If the predicted Hb value for the patient substantially matches the received Hb level value, the received DLCO value may be output. If the predicted Hb value for the patient does not substantially match the received Hb level value, a corrected DLCO value may be automatically calculated and output.

Some embodiments provide: means for receiving, from an Hb meter (e.g., a non-invasive Hb meter), an Hb level value for a patient; means for receiving, from a DLCO analyzer, a DLCO value for the patient; means for automatically calculating, by a computer, a predicted Hb value for the patient based on at least one patient parameter; if the predicted Hb value for the patient substantially matches the received Hb level value, means for outputting the received DLCO value; and if the predicted Hb value for the patient does not substantially match the received Hb level value, means for automatically calculating and outputting a corrected DLCO value.

A technical effect of some embodiments of the invention is an improved and computerized method to facilitate accurate DLCO measurements. With these and other advantages and features that will become hereinafter apparent, a more complete understanding of the nature of the invention can be obtained by referring to the following detailed description and to the drawings appended hereto.

### BRIEF DESCRIPTION OF THE DRAWINGS

FIG. 1 is a block diagram of an apparatus combining mini-box with DLCO and Hb according to some embodiments of the present invention.

FIG. 2 is DLCO and Hb flow diagram according to some embodiments of the present invention.

FIG. 3 is a block diagram of a system according to some embodiments of the present invention.

FIG. 4 illustrates a method that might be performed in accordance with some embodiments.

FIG. 5 is block diagram of a platform according to some embodiments of the present invention.

### DETAILED DESCRIPTION

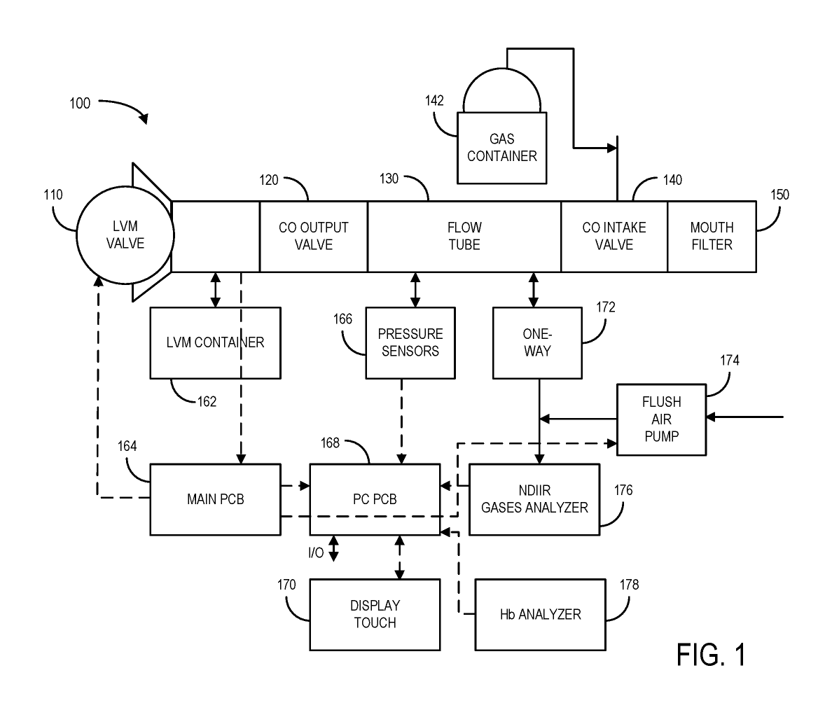

A significant number of patients undergoing a Diffusing capacity of the Lung for CO (“DLCO”) test may suffer from Anemia. Moreover, these patients may not have updated and accurate Hb results. This may lead to DLCO results that are significantly biased. That is, the readings may be much lower than normal. It would therefore be desirable to provide systems and methods to facilitate accurate DLCO measurements in an automated, efficient, and accurate manner. FIG. 1 is block diagram of a system 100 according to some embodiments of the present invention. In particular, the system includes a Lung Volume Measurement (“LVM”) valve 110, a CO output valve 120, a flow tube 130, a CO intake valve 140 (e.g., to receive an input from a gas container 142), and a mouth filter 150.

The system further includes an LVM container 162, a main PCB 164, pressure sensors 166, a PC PCB 168, and a display touch element 170. A one-way element 172 and flush air pump 174 may provide an input to a Non-Dispersive Infra-Red (“NDIR”) gases analyzer 176. An output of the NDIR gases analyzer 176 may be provided to the PC PCB 168 along with information from an Hb analyzer 178.

Since a lung doctor may want to distinguish between lung issues and Anemia issues, some embodiments described herein may measure both Hb and DLCO values simultaneously and calculate a DLCO value normalized to standard Hb values. Note that the formulas that correct the DLCO using the Hb results are suggested in the literature. One example is described in http://www.uptodate.com/contents/calculator-diffusing-capacity-for-carbon-monoxide-dlco-correction-of-predicted-value-for-anemia.

Some embodiments described herein integrate non-invasive Hb analyzer reading in real-time while a patient is performing a standard DLCO measurement. The results of the DLCO reading, Hb reading, and predicted DLCO results may be calculated and displayed. The non-invasive Hb analyzer may use, for example, spectroscopy techniques and might be measured on the patient's pointing finger. The DLCO measurements may be done according to the American Thoracic Society (“ATS”) recommendations. The predicted DLCO may be calculated, for example, using the preferred formula that suits the patient parameters (age, sex, weight, etc.). The calculation may be done automatically on a system computer and then be displayed along with the measured DLCO and measured Hb. According to some embodiments, integration with a MINIBOX may be provided as illustrated in FIG. 1, resulting in an all-in-one portable (or non-portable) device. Note that data acquired by the device might be associated with, for example, DLCO/Hb, Spirometry, and/or LVM. Further note that the MINIBOX illustrated in FIG. 1 is provided only as a non-limiting example, and embodiments may be associated with any other type of diagnostic Pulmonary Function Testing (“PFT”) device. For example, embodiments might be associated with a diagnostic device for pulmonary function testing that is a patient-friendly, portable and/or a desktop device designed to facilitate clinical decision making throughout diagnosis, treatment, and monitoring of respiratory diseases. Moreover, embodiments may be associated with any device for the measurement of lung parameters that is designed as either an all-in-one apparatus (such as, for example, a MINIBOX or MINIBOX+ available from PULMONE ADVANCED MEDICAL DEVICES LTD.) and/or a device that is associated with any combination of Spirometry and/or Lung Volume Measurement of total lung capacity (e.g., TLC, RV, VC, IC, etc.) and/or DLCO/Diffusing Capacity.

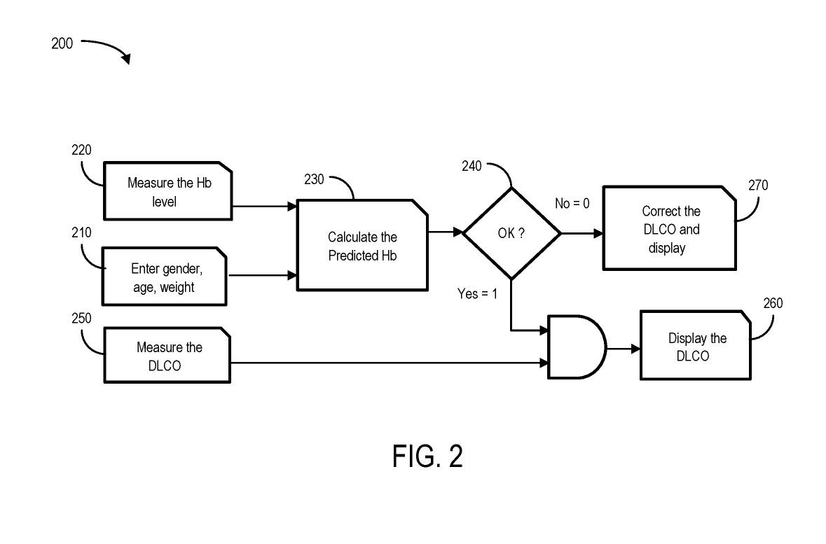

FIG. 2 is DLCO and Hb flow diagram 200 according to some embodiments of the present invention. A patient's age, gender, and weight 210 may be used along with a measured Hb level 220 to calculate a predicted Hb level 230. The predicted Hb level 230 may be compared 240 to the measured Hb level 220 for the patient. If the two values do not match, a measured DLCO value 250 may be corrected and displayed 270. If the two values agree, a measured DLCO value 250 may be output and displayed 260.

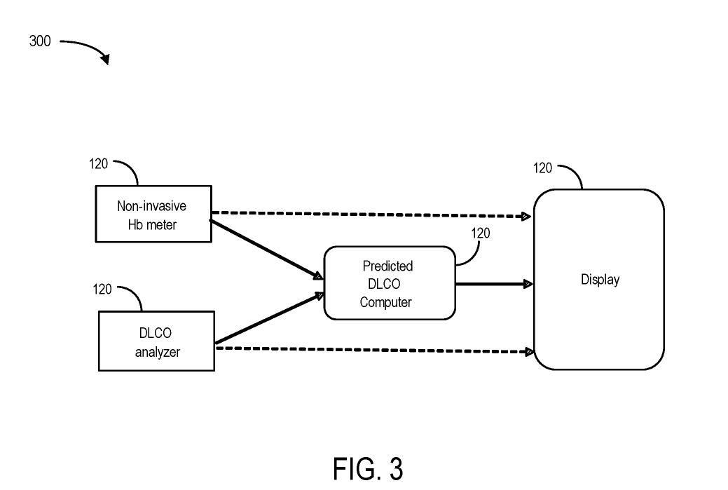

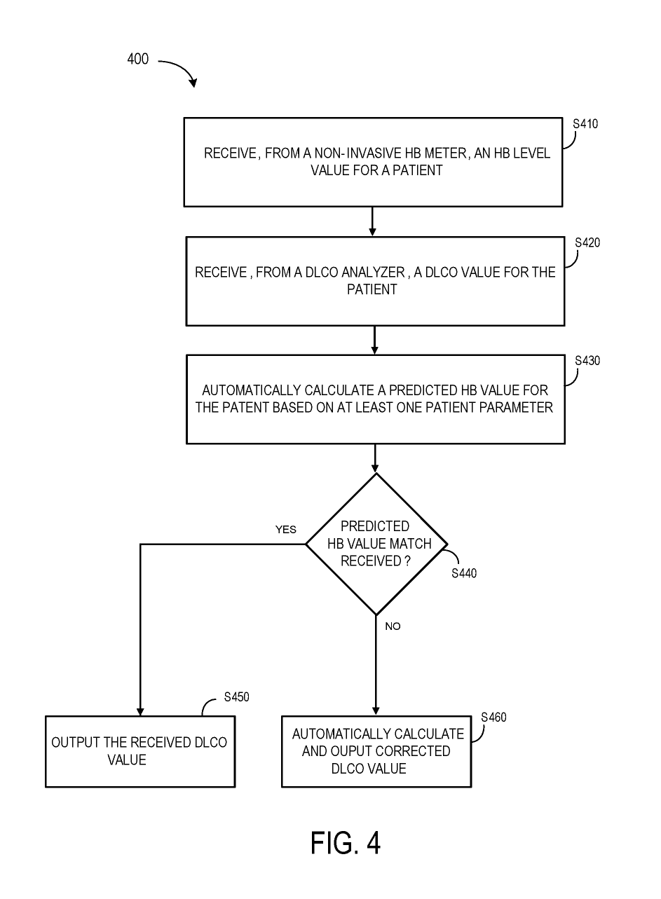

FIG. 3 is a block diagram of a system 300 according to some embodiments of the present invention. The system includes a non-invasive Hb meter 310, a DLCO analyzer 320, a predicted DLCO computer 330, and a display 340. FIG. 4 illustrates a method 400 that might be performed in accordance with some embodiments. The flow charts described herein do not imply a fixed order to the steps, and embodiments of the present invention may be practiced in any order that is practicable. Note that any of the methods described herein may be performed by hardware, software, or any combination of these approaches. For example, a computer-readable storage medium may store thereon instructions that when executed by a machine result in performance according to any of the embodiments described herein.

At S410, an Hb level value for a patient may be received from an Hb meter (e.g., a non-invasive Hb meter). At S420, a DLCO value for the patient may be received from a DLCO analyzer. A computer may then automatically calculate a predicted Hb value for the patient based on at least one patient parameter (e.g., age, gender, and/or weight) at S430. If the predicted Hb value for the patient substantially matches the received Hb level value at S440, the received DLCO value is output at S450. If the predicted Hb value for the patient does not substantially match the received Hb level value at S440, a corrected DLCO value is automatically calculated and output at S460.

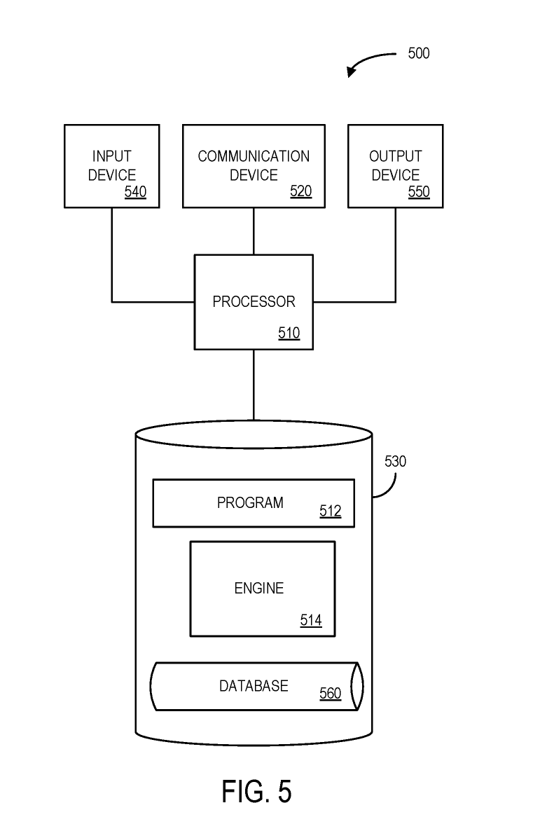

The embodiments described herein may be implemented using any number of different hardware configurations. For example, FIG. 5 illustrates a platform 500 that may be, for example, associated with any of the embodiments described here. The platform 500 comprises a processor 510, such as one or more commercially available Central Processing Units (CPUs) in the form of one-chip microprocessors, coupled to a communication device 520 configured to communicate via a communication network (not shown in FIG. 5). The communication device 520 may be used to communicate, for example, with one or more remote doctor or medical devices. The platform 500 further includes an input device 540 (e.g., a mouse and/or keyboard to enter information about a patient) and an output device 550 (e.g., to output a DLCO measurement or corrected DLCO value).

The processor 510 also communicates with a storage device 530. The storage device 530 may comprise any appropriate information storage device, including combinations of magnetic storage devices (e.g., a hard disk drive), optical storage devices, mobile telephones, and/or semiconductor memory devices. The storage device 530 stores a program 512 and/or an engine or application 514 for controlling the processor 510. The processor 510 performs instructions of the programs 512, 514, and thereby operates in accordance with any of the embodiments described herein. For example, the processor 510 may receive an Hb level value for a patient may be receiving from a non-invasive Hb meter. Moreover, a DLCO value for the patient may be received by the processor 510 from a DLCO analyzer at substantially the same time. The processor 510 may then automatically calculate a predicted Hb value for the patient based on at least one patient parameter (e.g., a patient gender, age, and/or weight). If the predicted Hb value for the patient substantially matches the received Hb level value, the received DLCO value may be output by the processor 510. If the predicted Hb value for the patient does not substantially match the received Hb level value, a corrected DLCO value may be automatically calculated and output by the processor 510.

The programs 512, 514 may be stored in a compressed, uncompiled and/or encrypted format. The programs 512, 514 may furthermore include other program elements, such as an operating system, a database management system, and/or device drivers used by the processor 510 to interface with peripheral devices.

As used herein, information may be “received” by or “transmitted” to, for example: (i) the platform 500 from another device; or (ii) a software application or module within the platform 500 from another software application, module, or any other source.

Thus, embodiments may provide an automated and efficient way to facilitate accurate DLCO measurements. The following illustrates various additional embodiments of the invention. These do not constitute a definition of all possible embodiments, and those skilled in the art will understand that the present invention is applicable to many other embodiments. Further, although the following embodiments are briefly described for clarity, those skilled in the art will understand how to make any changes, if necessary, to the above-described apparatus and methods to accommodate these and other embodiments and applications.

Although specific hardware and data configurations have been described herein, note that any number of other configurations may be provided in accordance with embodiments of the present invention (e.g., some of the information associated with the databases described herein may be combined or stored in external systems).

The present invention has been described in terms of several embodiments solely for the purpose of illustration. Persons skilled in the art will recognize from this description that the invention is not limited to the embodiments described, but may be practiced with modifications and alterations limited only by the spirit and scope of the appended claims.

What is claimed is:

1. A system to facilitate accurate DLCO measurements, the system comprising:
a communication device to receive, from a Hb meter, an Hb level value for a patient; and
a processor in communication with the communication device, wherein the processor is configured to:
receive, from a DLCO analyzer, a DLCO value for the patient,
automatically calculate a predicted Hb value for the patient based on at least one patient parameter,
if the predicted Hb value for the patient substantially matches the received Hb level value, output the received DLCO value, and
if the predicted Hb value for the patient does not substantially match the received Hb level value, automatically calculate and output a corrected DLCO value.

2. The system of claim 1, wherein the at least one patient parameter includes at least one of: (i) a patient gender, (ii) a patient age, and (iii) a patient weight.

3. A computerized method to facilitate accurate DLCO measurements, the method comprising:
receiving, from a non-invasive Hb meter, an Hb level value for a patient;
receiving, from a DLCO analyzer, a DLCO value for the patient;
automatically calculating, by a computer, a predicted Hb value for the patient based on at least one patient parameter;
if the predicted Hb value for the patient substantially matches the received Hb level value, outputting the received DLCO value; and
if the predicted Hb value for the patient does not substantially match the received Hb level value, automatically calculating and outputting a corrected DLCO value.

4. The method of claim 3, wherein the at least one patient parameter includes at least one of: (i) a patient gender, (ii) a patient age, and (iii) a patient weight.

5. A non-transitory, computer-readable medium storing instructions adapted to be executed by a computer processor to perform a method to facilitate accurate DLCO measurements, the method comprising:
receiving, from a non-invasive Hb meter, an Hb level value for a patient;
receiving, from a DLCO analyzer, a DLCO value for the patient;
automatically calculating, by a computer, a predicted Hb value for the patient based on at least one patient parameter;
if the predicted Hb value for the patient substantially matches the received Hb level value, outputting the received DLCO value; and
if the predicted Hb value for the patient does not substantially match the received Hb level value, automatically calculating and outputting a corrected DLCO value.

6. The medium of claim 5, wherein the at least one patient parameter includes at least one of: (i) a patient gender, (ii) a patient age, and (iii) a patient weight.
